(19) United States
(12) Patent Application Publication
(10) Pub. No.: US 2012/0114563 A1
(43) Pub. Date: May 10, 2012

(54) OPTICAL IMAGING AGENTS

(75) Inventors: Randall Lee Carter, Clifton Park, NY (US); Jason William Castle, Esperance, NY (US); Kenneth Michael Fish, Clifton Park, NY (US); Anup Sood, Clifton Park, NY (US); Natalie Anne Staples, Albany, NY (US); Brian Duh-Lan Lee, Rexford, NY (US)

(73) Assignee: GENERAL ELECTRIC COMPANY, SCHENECTADY, NY (US)

(21) Appl. No.: 13/257,318

(22) PCT Filed: Mar. 19, 2010
(86) PCT No.: PCT/EP10/53619

§ 371 Date: Jan. 10, 2012

Related U.S. Application Data
(60) Provisional application No. 61/161,535, filed on Mar. 19, 2009.

(51) Int. Cl. A61K 49/00; C07C 43/10

(52) U.S. Cl. 424/9.6; 568/672; 424/9.1

(57) ABSTRACT

The present invention relates to a method of in vivo optical imaging, of the margins around tumours, which comprises an optical imaging contrast agent. The optical imaging agents comprise conjugates of near-infrared dyes with synthetic polyethylene glycol (PEG) polymers having a molecular weight in the range 15-45 kDa. Also disclosed are optical imaging contrast agents, pharmaceutical compositions and kits.

### CROSS-REFERENCE TO RELATED APPLICATIONS

This application is a filing under 35 U.S.C. §371 and claims priority to international patent application number PCT/EP2010/053619 filed Mar. 19, 2010, published on Sep. 23, 2010 as WO 2010/106169, which claims priority to U.S. provisional patent application Ser. No. 61/161,535 filed Mar. 19, 2009.

### FIELD OF THE INVENTION

The present invention relates to a method of in vivo optical imaging, of the margins around tumours, which comprises an optical imaging contrast agent. The optical imaging agents comprise conjugates of near-infrared dyes with synthetic polyethylene glycol (PEG) polymers having a molecular weight in the range 15-45 kDa. Also disclosed are optical imaging contrast agents, pharmaceutical compositions and kits.

### BACKGROUND OF THE INVENTION

Despite great advances in scientific knowledge and the development of various therapeutic modalities, surgery remains the most frequently used and single most effective treatment of solid tumors in their early stages. Physically removing the tumor reduces symptoms, reduces the chance of the cancer spreading, decreases the amount of cancer in the body and helps other treatments to be more effective. Sixty to 70 percent of cancer patients will have surgery either by itself (40% of all cancers are treated with surgery alone), or in conjunction with other therapies usually radiation therapy or chemotherapy. Surgery is used to diagnose, stage, treat or manage complications during the course of disease in more than 90% of all cancer patients. Yet, while surgery is the oldest and most common form of cancer therapy, in many ways, it is also the least standardized intervention, in needs of new tools to help tracking diseased organs and differentiating normal and cancerous tissues. Surgeons traditionally depend on sight and touch (inspection and palpation) and any available pre-operative diagnostic imaging information to localize the tumor. Cancerous tissues are, however, often difficult to distinguish from normal tissues, or are too small to be detected (e.g. occult tumors). Thus, traditional surgical techniques do not ensure that all cancerous tissue has been found or removed and there is a need for agents, which can specifically identify cancer tissue, particularly tumor margins, with a very high resolution and sensitivity.

Wohrle et al [Makromol. Symp., 59, 17-33 (1992)] studied polymer-conjugation to porphyrin photosensitisers as a potential method of improving the uptake in target tissue in vivo for the photodynamic therapy of cancer. The polymers studied were rat serum albumin, synthetic polyethers and polyacohols. Wohrle et al concluded that the conjugation of a polymer carrier could improve the tumour uptake.

U.S. Pat. No. 5,622,685 discloses that polyether-substituted anti-tumour agents comprising a porphyrin, phthalocyanine or naphthalocyanine exhibit improved properties for both in vivo tumour diagnosis and therapy. The polyether substituents comprise polyethylene glycol (PEG) whose terminal hydroxyl group is etherified or esterified with C₁₋₁₂ alkyl or C₁₋₁₂ acyl groups respectively. The alkyl group is most preferably a methyl group. U.S. Pat. No. 5,622,685 teaches (column 2) that the total molecular weight of the conjugate is preferably at least 10,000 Da (10 kDa).

U.S. Pat. No. 6,083,485 and counterparts discloses in vivo near-infrared (NIR) optical imaging methods using cyanine dyes having an octanol-water partition coefficient of 2.0 or less. Also disclosed are conjugates of said dyes with “biological detecting units” of molecular weight up to 30 kDa which bind to specific cell populations, or bind selectively to receptors, or accumulate in tissues or tumours. The dyes of U.S. Pat. No. 6,083,485 may also be conjugated to a range of “non-selectively bonding” macromolecules, such as polylysine, dextran, carboxydextran, polyethylene glycol, methoxypolyethylene glycol, polyvinyl alcohol, or a cascade polymer-like structure. The molecular weight of the conjugates is taught to range from 100 Da to over 100,000 Da (0.1 to over 100 kDa). No specific dye-macromolecule conjugates are disclosed.

U.S. Pat. No. 6,350,431 (Nycomed Imaging AS) discloses light imaging contrast agents having a molecular weight in the range 500 to 500,000 Da, comprising a polyalkylene oxide (PAO) of molecular weight 60 to 100,000 Da having at least two chromophores (i.e. dye molecules) linked thereto. The polyalkylene oxide (PAO) moiety is taught to have a preferred molecular weight range of 200 to 100,000 Da, more preferably 250 to 50,000 Da, especially preferably 250 to 25,000 Da, most preferably 400 to 15,000 Da. The contrast agents of U.S. Pat. No. 6,350,431 may further comprise a targeting vector. The Examples of US 6,350,431 employ the following PAO polymers:
(i) PEG-diamine 3,400 Da molecular weight: Examples 1, 2, 6, 16, 18 and 25;
(ii) PEG-diamine 5,000 Da molecular weight: Examples 3, 4 and 20;
(iii) PEG-diamine 10,000 Da molecular weight: Examples 7, 15, 17 and 26;
(iv) PEG-dithiol 3,400 Da molecular weight: Example 12;
(v) PEG-dithiol 10,000 Da molecular weight: Example 13;
(vi) Poly(oxyethylene-co-oxypropylene-co-oxyethylene) block copolymer of average molecular weight about 14,600: Example 27.

Thus, the Examples of US 6,350,431 are all in the molecular weight range 3.4 to 14.6 kDa. For PEG polymers alone, the molecular weight range exemplified is 3.4 to 10 kDa.

Yuan et al [Cancer Res., 55, 3752-3756 (1995)] studied the vascular permeability of human tumour cells to dye-labelled macromolecules, and concluded that tumor vessels are in general more leaky and less permselective than normal cells. The tumour cell permeability was reported to vary twofold in the macromolecule molecular weight range 25 kDa to 160 kDa.

Dellian et al [Br. J. Cancer, 82(9), 1513-1518 (2000)] studied the effect of molecular charge on the vascular permeability of human tumour cells. They concluded that positively-charged molecules extravasate more quickly into solid tumours compared with neutral or negatively-charged compounds of similar molecular weight.

Licha et al [SPIE Vol 3196 p. 98-102 (1998)] disclose contrast agents for in vivo fluorescence imaging which comprise poly(ethyleneglycol) (PEG) polymers based on methoxypolyethylene glycol (MPEG). The conjugates thus have a heptamethine cyanine dye conjugated at one terminus of the PEG polymer and a methyl group at the other terminus:

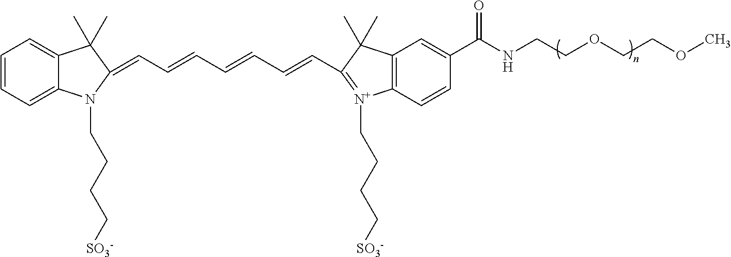

| Dye conjugate | n | Molecular weight (kDa) |
|---|---|---|
| NIR96017 | 22-28 | 1.83 |
| NIR96008 | 100-150 | 6.15 |
| NIR96486 | 240-320 | 13.2 |
| NIR96016 | 420-530 | 20.7 |

Also disclosed by Licha was a dye conjugate in which 2 MPEG chains were conjugated to a single cyanine dye (NIR96307, molecular weight ca. 41 kDa):

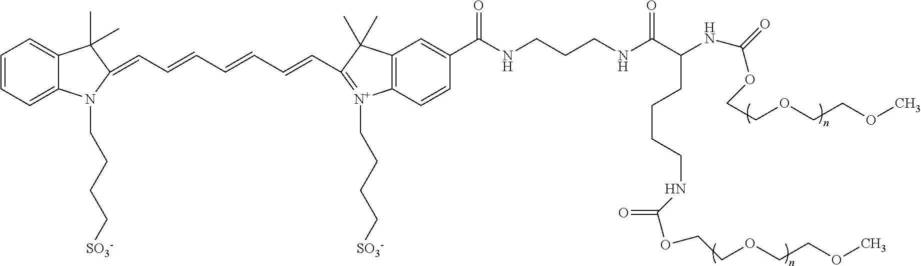

NIR96307

For NIR96307, n was not determined, but the mean molecular weight of the conjugate was said to be 41 kDa. The polymer conjugates of Licha were synthesized from the corresponding MPEG amine, ie. H₂NCH₂[CH₂OCH₂]ₙCH₂OCH₃.

In a related publication [Licha et al, SPIE Vol 3196, p. 103-110 (1998)] describe tumour detection in animals using the above MPEG conjugates. In particular, the interest was in the effect of the molecular weight of the PEG conjugate on: (i) their tolerability; (ii) the pharmacokinetic behaviour; and (iii) the contrast between malignant and normal tissue. They observed that increasing molecular weight prolonged the blood circulation time in vivo. They concluded that increased retention in the tumour environment and improved tumour contrast was observed at later times for dye-MPEG conjugates with a molecular weight above 6 kDa.

Montet et al [Radiology, 242(3), 751-758 (2007)] reported fluorescence molecular tomography (FMT) of angiogenesis using the near-infrared probes ANGIOSENSE® 680 and ANGIOSENSE® 750. These were described as high molecular weight (250 kDa) pegylated graft copolymers with an indocyanine-type fluorophore optimized for non-quenching. The agent contains MPEG attached to a polylysine backbone. Montet et al report that the agent exhibited a prolonged blood half-life (more than 5 hours), with no tumour extravasation up to 30 minutes post-administration, but increasing tumour uptake (and hence imaging brightness) with time thereafter.

Sadd et al [J. Control. Rel., 130, 107-114 (2008)] studied the characteristics of 3 different nanocarriers (linear polymer; dendrimers and liposome) on the efficacy of chemotherapy and imaging in vitro and in vivo. The linear polymer studied comprised a targeted PEG polymer of the type: [LHRH]-[PEG polymer]-Cy5.5 where: LHRH is a synthetic analogue of luteinizing hormone-releasing peptide; Cy5.5 is a specific cyanine dye.

The PEG polymer used had a molecular weight of about 3 kDa. FIG. 4 (p. 111) of Sadd et al compares the tumour uptake of the above conjugate with the non-targeted analogue, PEG-Cy5.5. Sadd et al concluded that the LHRH targeting polymer conjugate exhibits enhanced accumulation in cancer cells compared to the non-targeted analogue.

It is critical that curative surgery does not leave behind any tumor even of microscopic size. Residual and occult tumor tissue, undetectable during primary surgery, might evolve into a recurring cancer. That is why surgeons must ensure that no tumor has been left behind and the “margins” around the excised tumour are negative. Margins, also known as “margins of resection,” refer to the distance between a tumor and the edge of the surrounding tissue that is removed along with it. The excised tumor and surrounding tissue are subsequently examined by a pathologist in vitro. They are rolled in special ink so that the margins are clearly visible under a microscope. In clinical practice, the margins around a surgically-excised tumour are described as:
(i) positive margins: cancer cells extend out to the edge of the tissue, where the ink is;
(ii) negative margins: no cancer cells are found in the ink;
(iii) close margins: any situation that falls between positive and negative is to considered “close”.

Knowing how close cancer cells are to the edge of the excised tissue helps in making patient treatment decisions. If the margins are positive, additional surgery is needed. If the margins are close, surgery may or may not be needed or more surgery and the addition of radio- or chemo- therapy might be necessary. If the margins are negative, surgery is sufficient. The definition of “negative margins” varies from one hospital to another. In some places, if there is even one normal cell between the ink and the cancer cells, this is considered a negative margin. In other places, the pathologist will require at least two millimeters of tissue without cancer cells between the ink and the tumor before using the category “negative margins”. Typically, this analysis is performed after the surgery is complete so the identification of a “negative margin” before the patient has left the operating table would be of great benefit.

### SUMMARY OF THE INVENTION

The present invention provides a method of in vivo optical imaging of the margins around tumours, using an optical imaging contrast agent. The optical imaging agents comprise conjugates of near-infrared dyes with synthetic polyethylene glycol (PEG) polymers having a molecular weight in the range 15-45 kDa. Also disclosed are optical imaging contrast agents, pharmaceutical compositions and kits.

Using the MatBIII orthotropic rat breast cancer model and a prototype fluorescent image guided surgical system, the efficacy of the agents of the invention in highlighting tumour margins was determined Quantitation was achieved via a margin to surrounding skin ratio (MSR). Compared to actively targeting agents, the macromolecular passively-targeted agents gave improved results.

The present invention provides imaging agents capable of detecting sub-millimetre (down to 0.2-0.3 mm) foci of disease at the section level. The detection of the cancer foci can thus be achieved by the surgeon intraoperatively. The agent provides surgical guidance and/or identification of residual disease. Such imaging agents help to standardize surgery, irrespective of the volume of cancer patients operated by a surgeon and/or the experience of the pathologist. The agents help improve the efficiency of tumour surgery, maximising “negative margins” (as defined above), whilst minimising unnecessary excision of normal tissue from the patient.

### BRIEF DESCRIPTION OF THE DRAWINGS

FIG. 1 shows the effect of PEGs of differing molecular weights on the margin-to-skin ratio (MSR) of the imaging agent in a rat model of mammary gland adenocarcinoma. Further details are given in Example 3.

FIG. 2 shows the effect of different optical reporters on the margin-to-skin ratio (MSR) of the imaging agent in a rat model of mammary gland adenocarcinoma. Further details are given in Example 4.

### DETAILED DESCRIPTION OF THE INVENTION

In a first aspect, the present invention provides a method of in vivo optical imaging of the tumour margins of a tumour in an animate subject known to have at least one such tumour, said method comprising:
(i) providing an optical imaging contrast agent suitable for in vivo imaging, said contrast agent comprising a conjugate of a synthetic polyethylene glycol polymer of molecular weight 15 to 45 kDa, with one or two groups Opt^{R};
(ii) generating an optical image of a region of interest of said subject to which said contrast agent has been administered, said region of interest comprising said tumour and tumour margin;
   wherein each Opt^{R} is independently a biocompatible optical reporter group capable of detection either directly or indirectly in an optical imaging procedure using light of wavelength 600-850 nm.

By the term “optical imaging” is meant any method that forms an image for detection, staging or diagnosis of disease, follow up of disease development or for follow up of disease treatment based on interaction with light in the green to near-infrared region (wavelength 500-1200 nm). Optical imaging further includes all methods from direct visualization without use of any device and involving use of devices such as various scopes, catheters and optical imaging equipment, eg. computer-assisted hardware for tomographic presentations. The modalities and measurement techniques include, but are not limited to: luminescence imaging; endoscopy; fluorescence endoscopy; optical coherence tomography; transmittance imaging; time resolved transmittance imaging; confocal imaging; nonlinear microscopy; photoacoustic imaging; acousto-optical imaging; spectroscopy; reflectance spectroscopy; interferometry; coherence interferometry; diffuse optical tomography and fluorescence mediated diffuse optical tomography (continuous wave, time domain and frequency domain systems), and measurement of light scattering, absorption, polarization, luminescence, fluorescence lifetime, quantum yield, and quenching. Further details of these techniques are provided by: (Tuan Vo-Dinh (editor): “Biomedical Photonics Handbook” (2003), CRC Press LCC; Mycek & Pogue (editors): “Handbook of Biomedical Fluorescence” (2003), Marcel Dekker, Inc.; Splinter & Hopper: “An Introduction to Biomedical Optics” (2007), CRC Press LCC.

By the term “optical imaging contrast agent” is meant a compound suitable for optical imaging of a region of interest of the whole (ie. intact) mammalian body in vivo. Preferably, the mammal is a living human subject. The imaging may be invasive (eg. intra-operative or endoscopic) or non-invasive. The imaging is used to facilitate tumour resection (i.e. during intraoperative procedures) via tumour margin identification.

By the term “tumour margins” is meant the interstitial space on the periphery of the tumour between the lumen of the new tumour blood vessels and the tumour and normal cells surrounding the bulk of the tumour, wherein the leakiness of the new tumour blood vessels permits larger macromolecules to extravasate from the blood and get trapped or be temporarily concentrated in that interstitial area. This phenomenon is known as enhanced permeability and retention (EPR). Thus, cancer cells require additional nutrients to sustain their increased growth rates, and achieve this via angiogenesis. Angiogenesis is the process of new blood vessel formation. These new blood vessels also tend to have less structure than established vessels and are sometime termed “leaky” vasculature in that the junctions between the endothelial cells lining these vessels are not as tight and rigid as in established vessels. The angiogenic development of leaky microvasculature is common to all solid tumors [Folkman, Semin Cancer Biol., 3, 65-71 (1992) and Folkman, Nature Med., 1, 27-31 (1995)].

By the term “animate subject” is meant a living mammalian patient, preferably a living human subject.

The term “synthetic” has its conventional meaning, i.e. man-made as opposed to being isolated from natural sources. Such compounds have the advantage that their manufacture and impurity profile can be fully controlled.

The term “polyethylene glycol polymer” or “PEG” has its conventional meaning, as described eg. in “The Merck Index”, 14^{th} Edition entry 7568, i.e. a liquid or solid polymer of general formula H(OCH₂CH₂)ₙOH where n is an integer greater than or equal to 4. The polyethylene glycol polymers of the present invention may be linear or branched (i.e. dendrimeric), but are preferably linear. The polyethylene glycol polymer is suitably polydisperse. By the term “polymer terminus” is meant the functional group(s) which form the end of the polyether chains of the PEG polymer chains—in the above general formula the two hydroxy (—OH) groups.

By the term conjugate is “meant” a derivative in which the “optical reporter” (Opt^{R}) is covalently bonded to the polyethylene glycol polymer.

By the term “biocompatible” is meant non-toxic and hence suitable for administration to the mammalian body, especially the human body, without adverse reaction, or pain or discomfort on administration.

By the term “optical reporter” (i.e. Opt^{R}) is meant a fluorescent dye or chromophore which is capable of detection either directly or indirectly in an optical imaging procedure using light of wavelength 600-850 nm. Since the optical reporter must be suitable for imaging the mammalian body in vivo, it must also be biocompatible. Preferably, the Opt^{R} has fluorescent properties, and it preferably comprises a fluorescent, biocompatible dye.

The term “region of interest” or ROI has its conventional meaning in the field of in vivo medical imaging.

### Preferred Features.

The molecular weight of polyethylene glycol polymer is preferably 20-43 kDa, more preferably 22-40 kDa, and most preferably 25-38 kDa, with 27-35 kDa being the ideal. The polyethylene glycol polymer is preferably a linear polymer.

The polyethylene glycol polymer preferably only has conjugated thereto the Opt^{R} group(s). Thus, the polymer preferably does not have conjugated thereto a biological targeting molecule or other polymer. By the term “biological targeting moiety” is meant a compound which, after administration, is taken up selectively or localises at a particular site of the mammalian body. Such sites may for example be implicated in a particular disease state be indicative of how an organ or metabolic process is functioning. A biological targeting moiety typically comprises: 3-100 mer peptides, peptide analogue, peptoids or peptide mimetics which may be linear peptides or cyclic peptides or combinations thereof; or enzyme substrates, enzyme antagonists or enzyme inhibitors; synthetic receptor-binding compounds; oligonucleotides, or oligo-DNA or oligo-RNA fragments.

The conjugate of the first aspect is preferably of Formula I:

Y^{1l}—X^{a}-[POLYMER]—X^{b}—Y² (I)

where:
[POLYMER] is the synthetic polyethylene glycol polymer;
X^{a} and X^{b} are attached at the termini of said polyethylene glycol polymer, and are independently a bond or an L group;
where L is a linker group of formula -(A)ₘ- wherein each A is independently —CR₂—, —CR≡CR—, —CC—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid, or a sugar;
where each R is independently chosen from H, C₁₋₄ alkyl, C₂₋₄ alkenyl, C₂₋₄ alkynyl, C₁₋₄ alkoxyalkyl or C₁₋₄ hydroxyalkyl;
m is an integer of value 1 to 20;
Y¹ and Y² are independently Opt^{R} or a functional group chosen from —OH;
—O(C₁₋₁₀ alkyl); —NH₂ or —NH(CO)(C₁₋₁₀ alkyl);
wherein Opt^{R} is as defined above;
with the proviso that at least one of Y¹ and Y² is Opt^{R}.

By the term “amino acid” is meant an L- or D-amino acid, amino acid analogue (eg. naphthylalanine) or amino acid mimetic which may be naturally occurring or of purely synthetic origin, and may be optically pure, i.e. a single enantiomer and hence chiral, or a mixture of enantiomers.

By the term “sugar” is meant a mono-, di- or tri- saccharide. Suitable sugars include: glucose, galactose, maltose, mannose, and lactose. Optionally, the sugar may be functionalised to permit facile coupling to amino acids. Thus, eg. a glucosamine derivative of an amino acid can be conjugated to other amino acids via peptide bonds. The glucos amine derivative of asparagine (commercially available from

NovaBiochem) is one example of this:

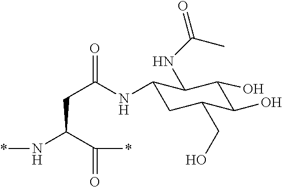

In Formula I, when only one of Y¹ and Y² is Opt^{R}, the other is preferably a functional group chosen from —OH and —NH₂, more preferably —OH.

In Formula I, it is preferred that each of Y¹ and Y² is Opt^{R}. In that instance, X and X′ are preferably chosen to be —NHCO— or —CONH— such that the conjugate is prepared from a diamino-PEG or dicarboxy-PEG polymer. Such PEG polymers thus correspond to H₂N-[POLYMER]—NH₂ or HOOC-[POLYMER]—COOH respectively, wherein the biocompatible dye of Opt^{R} is conjugated to the polymer at each terminus via an amide bond.

When each of Y¹ and Y² is Opt^{R}, it is preferred that the Opt^{R} groups of Y¹ and Y² each comprise the same biocompatible reporter. That has three advantages. Firstly, when the two chromophores of the biocompatible reporters are the same, the contrast agent exhibits an enhanced fluorescent signal for effectively the same molecular weight (because the molecular weight of the reporter is so much less than that of the polymer). Secondly, possible unwanted interference and/or quenching of fluorescence between the signals from two different biocompatible reporters is avoided. Thirdly, symmetric bifunctional-PEGs are easy to synthesise.

In Formula I, m of the L group is preferably an integer of value 1 to 5, most preferably 1 to 3.

The Opt^{R} preferably comprises a biocompatible dye capable of detection either directly or indirectly in an optical imaging procedure using light of wavelength 610-800 nm, more preferably 700-780 nm, most preferably 730-770 nm. The biocompatible dye of Opt^{R} preferably has fluorescent properties. Particular examples of such dyes include: indocyanine green, the cyanine dyes Cy5, Cy5.5, Cy7, and Alexa Fluor 633, Alexa Fluor 647, Alexa Fluor 660, Alexa Fluor 680, Alexa Fluor 700, and Alexa Fluor 750.

The biocompatible dye is preferably a cyanine dye or benzopyrylium dye, most preferably a cyanine dye. Preferred cyanine dyes which are fluorophores are of Formula II:

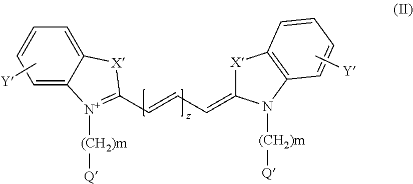

wherein:
each X′ is independently selected from: —C(CH₃)₂, —S—, —O— or —C[(CH₂)ₐCH₃][(CH₂)_{b}M]-, wherein a is an integer of value 0 to 5, b is an integer of value 1 to 5, and M is group G or is selected from SO₃M′ or H;
each Y′ independently represents 1 to 4 groups selected from the group consisting of: H, —CH₂NH₂, —SO₃M¹, —CH₂COOM¹, —NCS, F and a group G, and wherein the Y′ groups are placed in any of the positions of the aromatic ring;
Q′ is independently selected from the group consisting of: H, SO₃M¹, NH₂, COOM¹, ammonium, ester groups, benzyl and a group G;
M¹ is H or B^{c}; where B^{c} is a biocompatible cation;
z is an integer of value 2 or 3;
and m is an integer from 1 to 5;
wherein at least one of X′, Y′ and Q′ comprises a group G;
G is a reactive or functional group suitable for attaching to the PEG polymer.

By the term “biocompatible cation” (B^{c}) is meant a positively charged counterion which forms a salt with an ionised, negatively charged group, where said positively charged counterion is also non-toxic and hence suitable for administration to the mammalian body, especially the human body. Examples of suitable biocompatible cations include: the alkali metals sodium or potassium; the alkaline earth metals calcium and magnesium; and the ammonium ion. Preferred biocompatible cations are sodium and potassium, most preferably sodium.

The G group reacts with a complementary group of the PEG polymer forming a covalent linkage between the cyanine dye fluorophore and the polymer. The location of the G groups in Formula II is such that the PEG can suitably be conjugated at positions, Q′, X′ or Y′. G may be a reactive group that may react with a complementary functional group of the PEG, or alternatively may include a functional group that may react with a reactive group of the PEG. Examples of reactive and functional groups include: active esters; isothiocyanate; maleimide; haloacetamide; acid halide; hydrazide; vinylsulfone; dichlorotriazine; phosphoramidite; hydroxyl; amino; sulfydryl; carbonyl; carboxylic acid and thiophosphate. Preferably G is an active ester.

By the term “activated ester” or “active ester” is meant an ester derivative of the associated carboxylic acid which is designed to be a better leaving group, and hence permit more facile reaction with nucleophile, such as amines. Examples of suitable active esters are: N-hydroxysuccinimide (NHS), sulfo-succinimidyl ester, pentafluorophenol, pentafluorothiophenol, para-nitrophenol, hydroxybenzotriazole and PyBOP (i.e. benzotriazol- 1 -yl-oxytripyrrolidinophosphonium hexafluorophosphate). Preferred active esters are N-hydroxysuccinimide or pentafluorophenol esters, especially N-hydroxysuccinimide esters.

### Preferred Features of the Cyanine Dye.

Preferred cyanine dyes based on Formula II are as defined in Formula IIa:

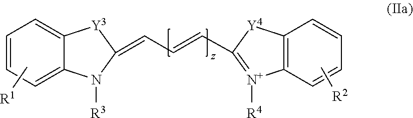

where:
Y³ and Y⁴ are independently —O—, —S—, —NR⁵— or —CR⁶R⁷— and are chosen such that at least one of Y³ and Y⁴ is —CR⁶R⁷—;
R¹ and R² are independently H, —SO₃M¹ or R^{a};
R³ to R⁵ are independently C₁₋₅ alkyl, C₁₋₆ carboxyalkyl or R^{a};
R⁶ is H or C₁₋₃ alkyl;
R⁷ is R^{a} or C₁₋₆ carboxyalkyl;
R^{a} is independently C₁₋₄ sulfoalkyl;
where M¹ and z are as defined in Formula II;

with the proviso that the cyanine dye of Formula IIa comprises at least one R^{a} group and a total of 1 to 6 sulfonic acid substituents from the R¹, R² and R^{a} groups.

By the term “sulfonic acid substituent” is meant a substituent of formula —SO₃M¹, where M¹ is as defined above. Preferred dyes of Formula IIa have z=3. Preferred such dyes also have 2 to 6 sulfonic acid substituents. The —SO₃M¹ substituent is covalently bonded to a carbon atom, and the carbon atom may be aryl (such as the R¹ or R² groups), or alkyl (i.e. an R^{a} group). In Formula IIa, the R^{a} groups are preferably of formula —(CH₂)ₖSO₃M¹, where M¹ is as defined above, and k is an integer of value 1 to 4. k is preferably 3 or 4. Cyanine dyes which are more preferred in Formula Ha have z =3, i.e. are heptamethine cyanine dyes.

Particularly preferred cyanine dyes are of Formula IIb:

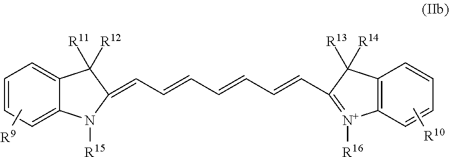

where:
R⁹ and R¹⁰ are independently H or SO₃M¹, and at least one of R⁹ and R¹⁰ is SO₃M¹;
R¹¹ and R¹² are independently C₁₋₄ alkyl or C₁₋₆ carboxyalkyl;
R¹³, R¹⁴, R¹⁵ and R¹⁶ are independently R^{b} groups;
wherein R^{b} is C₁₋₄ alkyl, C₁₋₆ carboxyalkyl or —(CH₂)_{q}SO₃M¹, where q is an integer of value 3 or 4;
where M¹ is as defined for Formulae II and IIa;
with the proviso that the cyanine dye has a total of 1 to 4 SO₃M¹
substituents in the R⁹, R¹⁰ and R^{b} groups.

Preferred cyanine dyes of Formula IIb are chosen to comprise at least one C₁₋₆ carboxyalkyl group, or activated ester thereof, in order to facilitate conjugation to the PEG polymer. An especially preferred such dye of Formula IIb is Cy7:

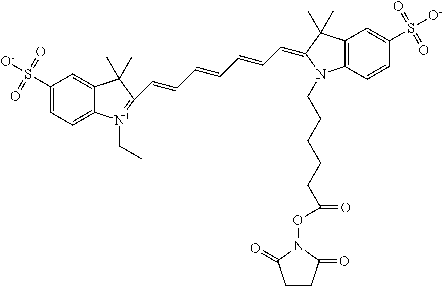

### Cy7-NHS Ester

The term “benzopyrylium dye” has its conventional meaning. Suitable benzopyrylium dyes of the present invention are denoted Bzp^{M} and are of Formula III:

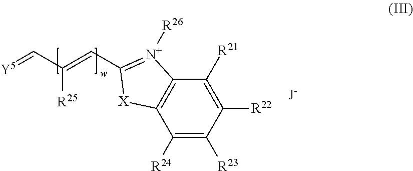

where:

Y⁵ is a group of Formula Y^{a} or Y^{b}

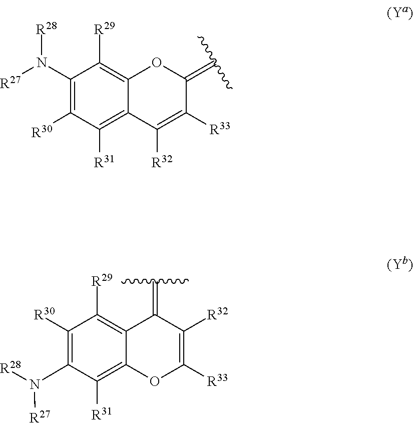

X is —CR³⁴R³⁵—, —O—, —S—, —Se—, —NR³⁶— or —CH═CH—, where R³⁴ to R³⁶ are independently R^{g} groups;

R²¹-R²⁴ and R²⁹-R³³ independently ndependently selected from H, —SO₃M¹, Hal, R^{g} or C₃₋₁₂ aryl;

R²⁵ is H, C₁₋₄ alkyl, C₁₋₆ carboxyalkyl, C₃₋₁₂ arylsulfonyl, Cl, or R²⁵ together with one of R²⁶, R³⁴, R³⁵ or R³⁶ may optionally form a 5- or 6- membered unsaturated aliphatic, unsaturated heteroaliphatic or aromatic ring;

R²⁶ and R³⁶ are independently Rg groups;

R²⁷ and R²⁸ are independently C₁₋₄ alkyl, C₁₋₄ sulfoalkyl or C₁₋₆ hydroxyalkyl or for Y^{a} may optionally together with one or both of R²⁹ and/or R³⁰ may form a 5- or 6- membered N-containing heterocyclic or heteroaryl ring, or for Y^{b} may optionally together with one or both of R³⁰and/or R³⁰ may form a 5- or 6- membered N-containing heterocyclic or heteroaryl ring;

R^{g} is C₁₋₄ alkyl, C₁₋₄ sulfoalkyl, C₁₋₆ carboxyalkyl or C₁₋₆ hydroxyalkyl;

w is 1 or 2;

J is a biocompatible anion;

where M¹ is as defined for Formula II;

with the proviso that Bzp^{M} comprises at least one sulfonic acid substituent chosen from the R²¹ to R³⁶ groups.

By the term “biocompatible anion” (J) is meant a negatively charged counterion which forms a salt with an ionised, positively charged group (in this case an indolinium group), where said negatively charged counterion is also non-toxic and hence suitable for administration to the mammalian body, especially the human body. The counterion (J⁻) represents an anion which is present in a molar equivalent amount, thus balancing the positive charge on the Bzp^{M} dye. The anion (J) is suitably singly- or multiply-charged, as long as a charge-balancing amount is present. The anion is suitably derived from an inorganic or organic acid. Examples of suitable anions include: halide ions such as chloride or bromide; sulfate; nitrate; citrate; acetate; phosphate and borate. A preferred such anion is chloride.

Suitable contrast agents of the invention are those wherein the Bzp^{M} is of Formula Ma or Mb:

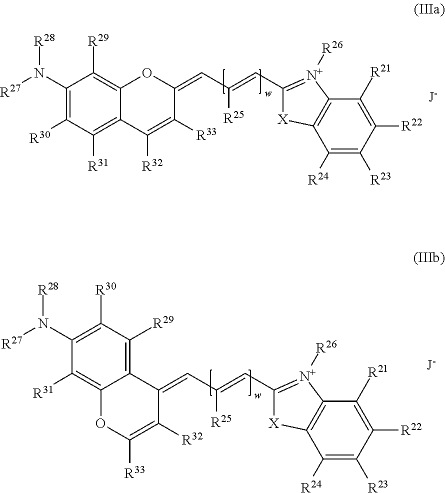

where X, w, J and R²¹-R³³ are as defined for Formula III.

When R²⁵ together with one of R²⁶/R³⁴-R³⁶ forms a 5- or 6- membered unsaturated aliphatic, unsaturated heteroaliphatic or aromatic ring, suitable such aromatic rings include: phenyl, furan, thiazole, pyridyl, pyrrole or pyrazole rings. Suitable unsaturated rings comprise at least the C≡C to which R²⁵ is attached.

When R²⁷ and/or R²⁸ together with at least one of R²⁹, R³⁰ or R³¹ (depending on whether Y¹ is Y^{a} or Y^{b} as described above), form a 5- or 6- membered N-containing heterocyclic or heteroaryl ring, suitable such rings include: thiazole, pyridyl, pyrrole or pyrazole rings or partially hydrogenated versions thereof.

preferably pyridyl or dihydropyridyl.

Preferred features of the Benzopyrylium Dye.

The PEG polymer is preferably attached at positions R²⁵, ^{R26}, R³⁴, R³⁵ or R³⁶ of the Bzp^{M} of Formula III, more preferably at R²⁶, R³⁴, R³⁵ or R³⁶ most preferably at R²⁶, R³⁴ or R³⁵. In order to facilitate the attachment the relevant R²⁵, R²⁶, R³⁴, R³⁵ or R³⁶ substituent preferably comprises C₁-₆ carboxyalkyl, more preferably C₃₋₆ carboxyalkyl.

The benzopyrylium dye (Bzp^{M}) preferably has at least 2 sulfonic acid substituents, more preferably 2 to 6 sulfonic acid substituents, most preferably 2 to 4 sulfonic acid substituents. Preferably, at least one of the sulfonic acid substituents is a C₁₋₄ sulfoalkyl group. Such sulfoalkyl groups are preferably located at positions R²⁶, R²⁷, R²⁸, R³⁴, R³⁵ or R³⁶; more preferably at R²⁶, R²⁷, R²⁸, R³⁴ or R³⁵; most preferably at R²⁶ together with one or both of R²⁷ and R²⁸ of Formula III. The sulfoalkyl groups of Formula III, are preferably of formula —(CH₂)ₖSO₃M¹, where M¹ is H or B^{c}, k is an integer of value 1 to 4, and B^{c} is a biocompatible cation (as defined above). k is preferably 3 or 4.

In Formula III, w is preferably 2. R²⁵ is preferably H or C₁₋₄ carboxyalkyl, and is most preferably H. X is preferably —CR³⁴R³⁵— or —NR³⁶—, and is most preferably —CR³⁴R³⁵—. Especially preferred benzopyrylium dyes having w=2 are DY-750 and DY-752, which are commercially available from Dyomics GmbH.

In the method of the first aspect, the contrast agent preferably comprises a pharmaceutical composition of the conjugate, together with a biocompatible carrier. Such pharmaceutical compositions are as described in the third aspect (below). The method of the first aspect is preferably carried out intraoperatively, to assist a surgeon in resection of the tumour from said subject. A preferred optical imaging method of the sixth aspect is Fluorescence Reflectance Imaging (FRI). In FRI, the contrast agent of the present invention is administered to a subject to be diagnosed, and subsequently a tissue surface of the subject is illuminated with an excitation light - usually continuous wave (CW) excitation. The light excites the Opt^{R} of the contrast agent. Fluorescence from the contrast agent, which is generated by the excitation light, is detected using a fluorescence detector. The returning light is preferably filtered to separate out the fluorescence component (solely or partially). An image is formed from the fluorescent light. Usually minimal processing is performed (no processor to compute optical parameters such as lifetime, quantum yield etc.) and the image maps the fluorescence intensity. The contrast agent is designed to concentrate in the disease area, producing higher fluorescence intensity. Thus the diseased area produces positive contrast in a fluorescence intensity image. The image is preferably obtained using a CCD camera or chip, such that real-time imaging is possible.

The wavelength for excitation varies depending on the particular dye used. The apparatus for generating the excitation light may be a conventional excitation light source such as: a laser (e.g., ion laser, dye laser or semiconductor laser); an array of LEDs; halogen light source or xenon light source. Various optical filters may optionally be used to obtain the optimal excitation wavelength.

In a first embodiment, a preferred FRI method comprises the steps of:
(i) a tissue surface comprising the region of interest within the animate subject is illuminated with an excitation light;
(ii) fluorescence from the contrast agent, which is generated by excitation of the Opt^{R} is detected using a fluorescence detector;
(iii) the light detected by the fluorescence detector is optionally filtered to separate out the fluorescence component;
(iv) an image of said tissue surface is formed from the fluorescent light of steps (ii) or (iii).

In the method comprising steps (i)-(iv), the excitation light of step (i) is preferably continuous wave (CW) in nature.

In a second embodiment, the optical imaging preferably comprises FDPM (frequency-domain photon migration). This has advantages over continuous-wave (CW) methods where greater depth of detection of the dye within tissue is important [Sevick-Muraca et al, Curr. Opin. Chem. Biol., 6, 642-650 (2002)]. For such frequency/time domain imaging, it is advantageous if the Opt^{R} has fluorescent properties which can be modulated depending on the tissue depth of the lesion to be imaged, and the type of instrumentation employed. A preferred FDPM method comprises the steps of:
(a) exposing light-scattering biologic tissue having a heterogeneous composition, said tissue forming a region of interest of said animate subject, to light from a light source with a pre-determined time varying intensity to excite the contrast agent, the tissue multiply-scattering the excitation light;
(b) detecting a multiply-scattered light emission from the tissue in response to said exposing;
(c) quantifying a fluorescence characteristic throughout the tissue from the emission by establishing a number of values with a processor, the values each corresponding to a level of the fluorescence characteristic at a different position within the tissue, the level of the fluorescence characteristic varying with heterogeneous composition of the tissue; and
(d) generating an image of the tissue by mapping the heterogeneous composition of the tissue in accordance with the values of step (c).

The fluorescence characteristic of step (c) preferably corresponds to uptake of the contrast agent and preferably further comprises mapping a number of quantities corresponding to adsorption and scattering coefficients of the tissue before administration of said contrast agent. The fluorescence characteristic of step (c) preferably corresponds to at least one of fluorescence lifetime, fluorescence quantum efficiency, fluorescence yield and contrast agent uptake. The fluorescence characteristic is preferably independent of the intensity of the emission and independent of contrast agent concentration.

The quantifying of step (c) preferably comprises: (i) establishing an estimate of the values, (ii) determining a calculated emission as a function of the estimate, (iii) comparing the calculated emission to the emission of said detecting to determine an error, (iv) providing a modified estimate of the fluorescence characteristic as a function of the error. The quantifying preferably comprises determining the values from a mathematical relationship modelling multiple light-scattering behaviour of the tissue. The method of the first option preferably further comprises monitoring a metabolic property of the tissue in vivo by detecting variation of said fluorescence characteristic.

The contrast agents of the first aspect can be prepared as follows:

In order to facilitate conjugation of the Opt^{R} to the PEG polymer, the dye of the Opt^{R} suitably has attached thereto a reactive functional group (Q^{a}). The Q^{a} group is designed to react with a complementary functional group of the polymer, thus forming a covalent linkage between the dye and the polymer. Suitable Q^{a} groups may be selected from: carboxyl; activated esters; isothiocyanate; maleimide; haloacetamide; hydrazide; vinylsulfone, dichlorotriazine and phosphoramidite. Preferably, Q^{a} is: an activated ester of a carboxylic acid; an isothiocyanate; a maleimide; or a haloacetamide. Most preferably Q^{a} is an activated ester. Preferred aspects of such activated esters are as described above.

General methods for conjugation of cyanine dyes to biological molecules are described by Licha et al [Topics Curr. Chem., 222, 1-29 (2002); Adv. Drug Deliv. Rev., 57, 1087-1108 (2005)]. Methods for conjugating cyanine dyes to PEG polymers are taught by Licha et al [SPIE Vol 3196 p. 98-102 (1998)].

When the conjugate comprises two Opt^{R} groups, one at each terminus of the to PEG polymer, a preferred starting material is a diamino-PEG. As noted by Elbert et al, [Elbert & Hubbell; Biomacromol., 2, 430-441 (2001)], such diamino-PEG materials can be of low purity. For the conjugates of the present invention, the PEG-doa,ome is preferably of greater than 90% purity, more preferably of over 95% purity, most preferably of over 99% purity. The synthesis described by Elbert provides PEG-diamines of the required purity. Example 1 provides further details.

Cyanine dyes functionalised suitable for conjugation to peptides are commercially available from GE Healthcare Limited, Atto-Tec, Dyomics, Molecular Probes and others. Most such dyes are available as NHS esters. Methods of conjugating the linker group (L) to the polymer employ analogous chemistry to that of the dyes alone (see above), and are known in the art. Benzopyrylium dyes are commercially available from Dyomics GmbH, Winzerlaer Str. 2A, D-07745 Jena, Germany.

In a second aspect, the present invention provides a contrast agent suitable for in vivo optical imaging of the mammalian body which comprises the conjugate as defined in the first aspect. Preferred embodiments of the conjugate in the contrast agent are as described in the first aspect.

In a third aspect, the present invention provides a pharmaceutical composition which comprises the conjugate as defined in the first aspect, together with a biocompatible carrier. Preferred embodiments of the conjugate in the pharmaceutical composition are as described in the first aspect.

The “biocompatible carrier” is a fluid, especially a liquid, in which the imaging agent can be suspended or dissolved, such that the composition is physiologically tolerable, ie. can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is isotonic); an aqueous solution of one or more tonicity-adjusting substances (eg. salts of plasma cations with biocompatible counterions), sugars (e.g. glucose or sucrose), sugar alcohols (eg. sorbitol or mannitol), glycols (eg. glycerol), or other non-ionic polyol materials (eg. polyethylene glycols, propylene glycols and the like). When a macromolecular polyol is used, it is suitably of molecular weight up no more than 10 kDa, preferably below 5 kDa—since higher molecular weight species might compete with the contrast agent of the present invention. Preferably, the biocompatible carrier is pyrogen-free water for injection or isotonic saline.

The contrast agent and biocompatible carrier are each supplied in suitable vials or vessels which comprise a sealed container which permits maintenance of sterile integrity and/or radioactive safety, plus optionally an inert headspace gas (eg. nitrogen or argon), whilst permitting addition and withdrawal of solutions by syringe or cannula. A preferred such container is a septum-sealed vial, wherein the gas-tight closure is crimped on with an overseal (typically of aluminium). The closure is suitable for single or multiple puncturing with a hypodermic needle (e.g. a crimped-on septum seal closure) whilst maintaining sterile integrity. Such containers have the additional advantage that the closure can withstand vacuum if desired (eg. to change the headspace gas or degas solutions), and withstand pressure changes such as reductions in pressure without permitting ingress of external atmospheric gases, such as oxygen or water vapour.

Preferred multiple dose containers comprise a single bulk vial (e.g. of 10 to 30 cm³ volume) which contains multiple patient doses, whereby single patient doses can thus be withdrawn into clinical grade syringes at various time intervals during the viable lifetime of the preparation to suit the clinical situation. Pre-filled syringes are designed to contain a single human dose, or “unit dose” and are therefore preferably a disposable or other syringe suitable for clinical use. The pharmaceutical compositions of the present invention preferably have a dosage suitable for a single patient and are provided in a suitable syringe or container, as described above.

The pharmaceutical composition may optionally contain additional excipients such as an antimicrobial preservative, pH-adjusting agent, filler, stabiliser or osmolality adjusting agent. By the term “antimicrobial preservative” is meant an agent which inhibits the growth of potentially harmful micro-organisms such as bacteria, yeasts or moulds. The antimicrobial preservative may also exhibit some bactericidal properties, depending on the dosage employed. The main role of the antimicrobial preservative(s) of the present invention is to inhibit the growth of any such micro-organism in the pharmaceutical composition. The antimicrobial preservative may, however, also optionally be used to inhibit the growth of potentially harmful micro-organisms in one or more components of kits used to prepare said composition prior to administration. Suitable antimicrobial preservative(s) include: the parabens, ie. methyl, ethyl, propyl or butyl paraben or mixtures thereof; benzyl alcohol; phenol; cresol; cetrimide and thiomersal. Preferred antimicrobial preservative(s) are the parabens.

The term “pH-adjusting agent” means a compound or mixture of compounds useful to ensure that the pH of the composition is within acceptable limits (approximately pH 4.0 to 10.5) for human or mammalian administration. Suitable such pH-adjusting agents include pharmaceutically acceptable buffers, such as tricine, phosphate or TRIS [ie. tris(hydroxymethyl)aminomethanel, and pharmaceutically acceptable bases such as sodium carbonate, sodium bicarbonate or mixtures thereof. When the composition is employed in kit form, the pH adjusting agent may optionally be provided in a separate vial or container, so that the user of the kit can adjust the pH as part of a multi-step procedure.

By the term “filler” is meant a pharmaceutically acceptable bulking agent which may facilitate material handling during production and lyophilisation. Suitable fillers include inorganic salts such as sodium chloride, and water soluble sugars or sugar alcohols such as sucrose, maltose, mannitol or trehalose.

The pharmaceutical compositions may be prepared under aseptic manufacture (ie. clean room) conditions to give the desired sterile, non-pyrogenic product. It is preferred that the key components, especially the associated reagents plus those parts of the apparatus which come into contact with the imaging agent (eg. vials) are sterile. The components and reagents can be sterilised by methods known in the art, including: sterile filtration, terminal sterilisation using e.g. gamma-irradiation, autoclaving, dry heat or chemical treatment (e.g. with ethylene oxide). It is preferred to sterilise some components in advance, so that the minimum number of manipulations needs to be carried out. As a precaution, however, it is preferred to include at least a sterile filtration step as the final step in the preparation of the pharmaceutical composition.

The pharmaceutical composition is preferably prepared from a kit, as described for the fourth aspect below.

In a fourth aspect, the present invention provides a kit for the preparation of the pharmaceutical composition of the second aspect, which comprises the contrast agent of the first aspect in sterile, solid form such that, upon reconstitution with a sterile supply of a biocompatible carrier (as described in the third aspect), dissolution occurs to give the desired pharmaceutical composition.

In that instance, the contrast agent, plus other optional excipients as described above, may be provided as a lyophilised powder in a suitable vial or container. The agent is then designed to be reconstituted with the desired biocompatible carrier to give the pharmaceutical composition in a sterile, apyrogenic form which is ready for mammalian administration.

A preferred sterile, solid form of the contrast agent is a lyophilised solid. The sterile, solid form is preferably supplied in a pharmaceutical grade container, as described for the pharmaceutical composition (above). When the kit is lyophilised, the formulation may optionally comprise a cryoprotectant chosen from a saccharide, preferably mannitol, maltose or tricine.

### EXAMPLES

The present examples are provided for illustrative purposes only, and are not to be construed as limiting the present invention as defined by the appended claims. All references given below and elsewhere in the present specification are hereby included herein by reference.

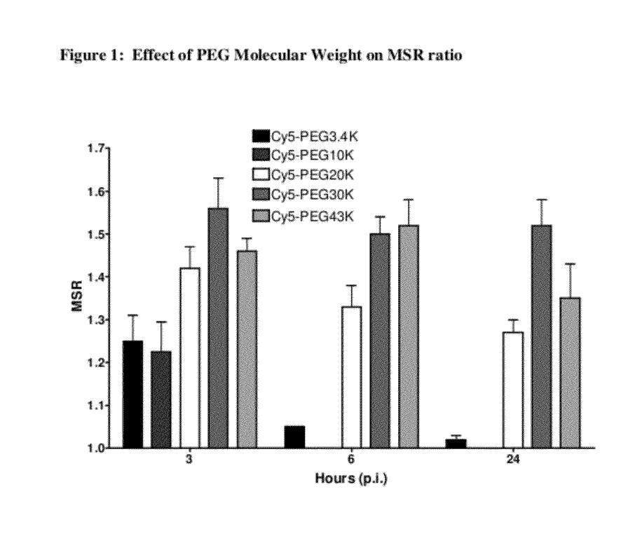

The invention is illustrated by the non-limiting Examples detailed below.
Example 1 provides the synthesis of a PEG-bis(dye) conjugate of the invention.
Example 2 provides the synthesis of other PEG-dye conjugates of the invention.
Example 3 provides the biological screening model used. The results are shown in FIG. 1. Earlier time points were regarded as being somewhat more important, since that is more pertinent to the clinical situation. The PEG3.4k conjugate has markedly inferior MSR values at all times. The PEG30k conjugate was superior, exhibiting good MSR values at all time points. The PEG20k and PEG43k conjugates exhibited good MSR values at earlier times, but were inferior at 24 hours.

Example 4 examined the effect of changing the dye from the Cy5 range (excitation: 650 nm, emission: 670 nm) to Cy7 (excitation: 743 nm, emission: 767 nm). For the Cy7 conjugate, the MSR score either increased or was similar Further evaluation of commercially available Cy7-analog dyes from Dyomics, DY752 and DY750, exhibited remarkably similar MSR scores as compared to Cy7.

### Abbreviations.

Conventional 3-letter and single letter amino acid abbreviations are used.
Acm: Acetamidomethyl
ACN: Acetonitrile
Boc: tert-Butyloxycarbonyl
DMF: N,N′-Dimethylformamide
DMSO: Dimethylsulfoxide
GFC: gel filtration chromatography
HCl: Hydrochloric acid
HPLC: High performance liquid chromatography
MALDI: Matrix assisted laser desorption ionization.
MSR: margin to surrounding skin ratio.
NHS: N-hydroxy-succinimide
PBS: Phosphate-buffered saline.
TFA: Trifluoroacetic acid.

### Example 1

### Synthesis of a Bis-Cy7 PEG-31k Conjugate (Compound 1)

Diamino-PEG was purchased from supplier LaysanBio. It was synthesised from the corresponding PEG-diol (Sigma/Aldrich), using the method of Elbert et al [Biomacromolecules, 2, p 430-441 (2001)]. The diamine-PEG had an average mass of ˜31 kDa by GFC and ˜35 kDa by MALDI. Amine substitution was ca. 100% with no other impurities detectable by proton NMR, in particular no CH₂-OMs or CH₂—OH protons observable.

The fluorescent dye, Cy7-NHS was obtained from GE Healthcare. It had an active ester content of 81.3%. The conjugate was prepared as follows:

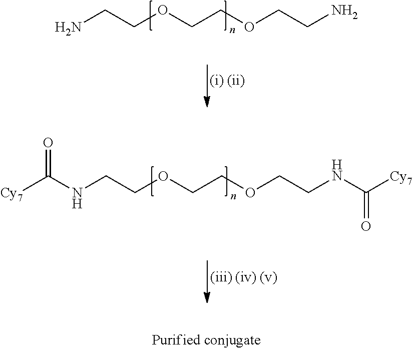

(i) dissolve diamino-PEG -31k (10 mg/ml) in 0.1 M NaHCO₃ buffer. Adjust pH to 8.5-8.8 with 1M NaOH;

(ii) add 3 equiv. Cy7-NHS solution (ca. 1 mg/100 μL in DMSO)—concentration measured by UV/VIS prior to use. Stirred at room temperature overnight;

(iii) preparative RP-LC using AKTA purifier;

(iv) concentrated at room temperature in vacuo, then either co-evaporated to dryness with water (x3), or lyophilised;

(v) formulated in PBS at 75 μM concentration.

The Cy5 conjugate, and the conjugates with the benzopyrylium dyes Dy750 and Dy752 (Dyomics GmbH, D-07745, Jena, Germany) were prepared in an analogous manner.

### Example 2

### Synthesis of Other PEG-dye Conjugates

PEGs functionalised with a single dye molecule were synthesised in an analogous manner to Example 1, using the appropriate PEG-monoamine with the dye active ester (˜1.2-1.5 equivalents).

The PEG 43 kDa conjugate was prepared by reaction of mono-amino PEG20K with a bifunctional dye (Cy5-bis NHS ester) in a molar ratio of 3.33:1. Thus, PEG20K (100 mg) was co-evaporated with anhydrous DMF (3x) and redissolved in anhydrous DMF (5 ml). To this solution N-methylmorpholine (4 nl) was added followed by a solution of Cy5-bis NHS (0.3 equiv. in 146 μl of DMSO). The mixture was stirred in the dark overnight and then purified by HPLC. The pure fraction was concentrated using Amicon 5K MWCO filter.

### Example 3

### Screening Model

All cell lines were obtained from the American Type Culture Collection (ATCC, Manassas, VA) and cultured as recommended. The 13762 Mat B III (a rat mammary gland adenocarcinoma; ATCC # CRL-1666) cell line was cultured in DMEM (Gibco #10564-011) with 10% FBS and 1% Pen/Strep. Cells were incubated at 37° C. in a mixture of air:CO₂ (95%:5%). After cells reached more than 80% confluency, cells were collected, counted, and concentrated to 10×10⁶ cells/mL of culture media for injection.

Animals and in vivo Tumour Models.

In vivo studies were carried out with female Fischer 344 rats or severe combined immunodeficient (SCID) mice, each with an age between 4 and 8 weeks Animals were housed with non-fluorescent food (Harlan Labs, cat. #TD.97184) and water ad libitum and a standard 12 hour day-night lighting cycle. 1×10⁶ cells (100uL) were injected, using a 27 gauge needle, directly orthotopically into the mammary fat pad of the animal. After allowing 7 days for MatBIII tumor growth (˜1 cm in diameter), animals were injected with test agent and imaged.

Imaging.

Images of the animals were taken at the shortest exposure time of 60 ms to minimize motion artifacts with a gain of 250. Images were analyzed using analysis software. The margin was automatically selected and a margin area of 41 pixels outside of the margin was highlighted. The result was discrimination of the margin and the tumour from the background of the image. A margin-to-skin ratio (MSR) is calculated using the equation below:

$$M S R = {( \frac{{I}_{margin}}{{I}_{background}} )}^{2}$$

FIG. 1 shows the effect of PEGs of differing molecular weights. The number of each PEG represents the molecular weight of the agent (i.e. PEG3OK has a MW of 30 kDa).

### Example 4

### Effect of Dye

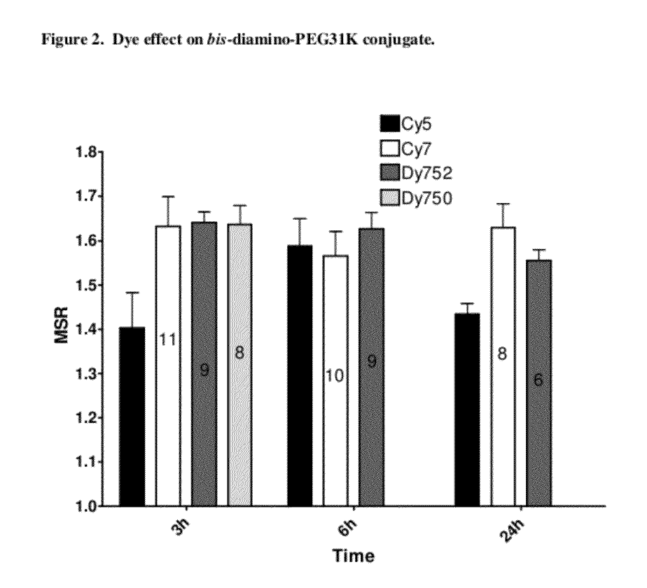

Different dyes with longer excitation and emission wavelengths were conjugated to the bis-diamino-PEG31K backbone as described in Example 1. The MSR results are shown in FIG. 2.

The above mentioned examples of conceivable embodiments are intended to illustrate the present invention and are not intended to limit the scope of protection claimed by the following claims.

1. A method of in vivo optical imaging of the tumour margins of a tumour in an animate subject known to have at least one such tumour, said method comprising:
(i) providing an optical imaging contrast agent suitable for in vivo imaging, said contrast agent comprising a conjugate of a synthetic polyethyleneglycol polymer of molecular weight 15 to 45 kDa, with one or two groups Opt^{R};
(ii) generating an optical image of a region of interest of said subject to which said contrast agent has been administered, said region of interest comprising said tumour;
wherein each Opt^{R} is independently a biocompatible optical reporter group capable of detection either directly or indirectly in an optical imaging procedure using light of wavelength 600-850 nm.

2. The method of claim 1, where the polymer has conjugated thereto only the Opt^{R} group(s).

3. The method of claim 1 or claim 2, where the conjugate is of Formula I:
Y¹—X^{a}-[POLYMER]—X^{b}—Y² (I)
where:
[POLYMER] is the synthetic polyethyleneglycol polymer;
X^{a} and X^{b} are attached at the termini of said polyethyleneglycol polymer, and are independently a bond or an L group;
where L is a linker group of formula -(A)ₘ- wherein each A is independently —CR₂—, —CR═CR—, —C≡C—, —CR₂CO₂—, —CO₂CR₂—, —NRCO—, —CONR—, —NR(C═O)NR—, —NR(C═S)NR—, —SO₂NR—, —NRSO₂—, —CR₂OCR₂—, —CR₂SCR₂—, —CR₂NRCR₂—, a C₄₋₈ cycloheteroalkylene group, a C₄₋₈ cycloalkylene group, a C₅₋₁₂ arylene group, or a C₃₋₁₂ heteroarylene group, an amino acid, or a sugar;
where each R is independently chosen from H, C₁₋₄ alkyl, C₂₋₄ alkenyl, C₂₋₄ alkynyl, C₁₋₄ alkoxyalkyl or C₁₋₄ hydroxyalkyl;
m is an integer of value 1 to 20;
Y¹ and Y² are independently Opt^{R} or a functional group chosen from —OH; —O(C₁₋₁₀ alkyl); —NH₂ or —NH(CO)(C₁₋₁₀ alkyl);
wherein Opt^{R} is as defined in claim 1;
with the proviso that at least one of Y¹ and Y² is Opt^{R}.

4. The method of claim 3, where each of Y¹ and Y² is Opt^{R}.

5. The method of claim 4, where the Opt^{R} groups of Y¹ and Y² each comprise the same biocompatible optical reporter.

6. The method of any one of claims 1 to 5, where the biocompatible optical reporter is a cyanine dye.

7. The method of any one of claims 1 to 5, where the biocompatible optical reporter group is a benzopyrylium dye.

8. The method of any one of claims 1 to 7, where the polyethyleneglycol polymer has a molecular weight of 22 to 40 kDa.

9. The method of any one of claims 1 to 8, where the polyethyleneglycol polymer is a linear polymer.

10. The method of any one of claims 1 to 9, where the contrast agent comprises a pharmaceutical composition of the conjugate as defined in any one of claims 1 to 9, together with a biocompatible carrier.

11. The method of any one of claims 1 to 10, which comprises the steps of:
(i) a tissue surface comprising the region of interest within the animate subject, as defined in claim 1, is illuminated with an excitation light;
(ii) fluorescence from the contrast agent, which is generated by excitation of the Opt^{R} is detected using a fluorescence detector;
(iii) the light detected by the fluorescence detector is optionally filtered to separate out the fluorescence component;
(iv) an image of said tissue surface is formed from the fluorescent light of steps (ii) or (iii).

12. The method of claim 11 where the excitation light of step (i) is continuous wave (CW) in nature.

13. The method of any one of claims 1 to 10, which comprises:
(a) exposing light-scattering biologic tissue having a heterogeneous composition, said tissue forming a region of interest of said animate subject, to light from a light source with a pre-determined time varying intensity to excite the contrast agent, the tissue multiply-scattering the excitation light;
(b) detecting a multiply-scattered light emission from the tissue in response to said exposing;
(c) quantifying a fluorescence characteristic throughout the tissue from the emission by establishing a number of values with a processor, the values each corresponding to a level of the fluorescence characteristic at a different position within the tissue, the level of the fluorescence characteristic varying with heterogeneous composition of the tissue; and
(d) generating an image of the tissue by mapping the heterogeneous composition of the tissue in accordance with the values of step (c).

14. The method of any one of claims 1 to 13, where the optical imaging is carried out intraoperatively, to assist a surgeon in resection of the tumour from said subject.

15. A contrast agent suitable for in vivo optical imaging of the mammalian body which comprises the conjugate as defined in any one of claims 1 to 9.

16. A pharmaceutical composition which comprises the conjugate as defined in any one of claims 1 to 9, together with a biocompatible carrier.

17. The pharmaceutical composition of claim 16, which has a dosage suitable for a single patient and is provided in a suitable syringe or container.

18. A kit for the preparation of the pharmaceutical composition of claim 16 or claim 17, which comprises the conjugate as defined in any one of claims 1 to 9 in sterile, solid form such that upon reconstitution with a sterile supply of the biocompatible carrier, dissolution occurs to give the desired pharmaceutical composition.

19. The kit of claim 18, where the sterile, solid form is a lyophilised solid.
